(19) United States
(12) Patent Application Publication
(10) Pub. No.: US 2005/0215952 A1
(43) Pub. Date: Sep. 29, 2005

(54) Disposable injection device designed to be pre-filled

(75) Inventors: Marc Brunel, Toulouse (FR); Lucien Cany, Pont de Larn (FR)

Correspondence Address: YOUNG & THOMPSON, 745 SOUTH 23RD STREET, 2ND FLOOR, ARLINGTON, VA, 22202, US

(73) Assignee: MB INNOVATION, TOULOUSE (FR)

(21) Appl. No.: 10/515,476

(22) PCT Filed: May 26, 2003
(86) PCT No.: PCT/FR03/01588

§ 371 Date: Nov. 24, 2004

(30) Foreign Application Priority Data
May 27, 2002 (FR) 02/06446

(51) Int. Cl. A61M 5/00

(52) U.S. Cl. 604/111; 604/198

(57) ABSTRACT

A single-use injection device includes a tubular receptacle provided with sealingly closing-off elements delimiting a chamber intended to be filled with a liquid, and having a liquid-delivery duct opening into the chamber, a needle-carrying base having a duct for supplying liquid to the needle and being axially movable between a retracted position in which the delivery duct is closed off, and an advanced injection position in which it allows the liquid to flow out of the chamber to the needle, a cap having a shape adapted to cover the base and provided with elements for detachable connection to the tubular receptacle, axial-stop elements arranged so as to keep the base in its closing-off position when the latter is covered by the cap, and axial-stop elements arranged so as to limit the axial-displacement travel of the base, once the cap is removed.

The invention relates to an injection device of the single-use type, which is designed to be pre-filled with a dose of liquid, in particular a medicinal liquid, to be injected.

Single-use injection devices which are intended to be pre-filled comprise a syringe body, which is either provided with a base in which there is embedded a needle which is protected by a protective cap, or comprise a conical male locking connection which makes it possible to fit onto the said syringe body a conical female locking connection, carrying an injection needle protected by a protective cap, the said conical connections defining an assembly which is commonly known as a “LÜER conical assembly”.

The most conventional injection devices, known as “wet needle” devices, are of the type provided with a syringe body comprising a base in which there is embedded a needle closed off by means of a protective elastomer cap, which is provided with a inner blind bore arranged in the base of the said cap, and inside which the end of the said needle is forced, so as to guarantee the sealing of the injection device before injection.

The first disadvantage of injection devices of this type consists in the fact that when the protective cap is put into place, the injection needle must be centred perfectly in relation to the bore in the said cap. However, in practice, this centring is sometimes approximate, so that fitting of the cap frequently leads to damage of the said cap or needle, resulting in significant numbers of production rejects.

In addition, according to this principle, the quality of the needle (sharpness, siliconing), is systematically affected by friction of the point of the said needle against the inner wall of the blind bore in the cap, when the latter is forced on.

Finally, the liquid contained in these injection devices is necessarily in contact with the materials which constitute the injection needle and the protective cap, which, for certain types of liquid, can affect the conservation of the latter.

In order to overcome these disadvantages, numerous injection devices known as “dry needle” devices have been designed, wherein the injection needle is isolated from the liquid contained in the syringe body until the moment of injection.

A first type of “dry needle” injection device is that which is commonly used in dentistry, and comprises a bottle, containing the liquid to be injected and closed off by a membrane, and a double-point needle which can be displaced axially relative to the said bottle so as to pierce the membrane at the moment of injection. Injection devices of this type are described in particular in patents DE-847473, FR-2347055, U.S. Pat. No. 4,639,250, EP-602883, DE-2008751, DE-1909794.

This type of injection device has two sorts of disadvantages. Firstly, the fact of that a double-point needle is required leads to an increase in the cost price of these injection devices, derived on the one hand from the cost of the said needle itself, and on the other hand from the necessity to carry out two sharpening operations instead of a single sharpening operation required for a conventional needle. In addition, for needles with a small diameter, it often happens that problems of coring arise, leading to the inclusion of particles of membrane inside the aperture of the needle, which either close off this aperture, or are injected together with the liquid.

A second type of “dry needle” injection device comprises a syringe body which accommodates two stoppers, delimiting a chamber containing the liquid, and onto which is crimped a base either carrying a needle or of the conical male connection type, the said syringe body furthermore having a compartment which is provided with a duct for communication with the injection needle, which is arranged so as to be put into contact with the chamber only after the plungers have been displaced axially.

Injection devices of this type, which are described in particular in patents FR-2412320, FR-2208684, EP-191508, EP-588148 and EP-720857, make it possible to overcome the disadvantages of the above-described injection devices. However, they themselves also have two disadvantages. Firstly, the operation of crimping the base onto the syringe body is problematic, and requires particular care in order to guarantee perfect sealing between the said base and the said syringe body. In addition, and above all, injection devices of this type can be subject to accidental escape of the liquid contained in the chamber, resulting for example from expansion of the volume of gas contained in the said chamber, or from depressurisation, in particular during transport by air, which lead to axial displacement of the stopper for access to the compartment for discharge of the liquid.

A third type of “dry needle” injection device, described in particular in patents EP-150681, EP-111796, FR-2330413, WO-8404252, FR-2784033, makes it possible to overcome all of the above-described disadvantages. For this purpose, these injection devices comprise, on the one hand, a syringe body which is provided with a chamber closed off by a rubber stopper containing a longitudinal through-bore, and on the other hand, a base, either carrying a needle or of the conical male connection type, which is movable axially inside the bore of the said stopper and is provided with ducts which are arranged so as to put the injection needle and the chamber into communication during axial displacement of the said base, which tends to thrust the latter into the stopper.

However, such injection devices in practice have a major disadvantage resulting from the fact that their activation, for the purpose of an injection, requires thrusting the base inside the protective stopper. It follows from this that the user is forced to perform a specific action aimed at bringing about the activation of the injection device entailing modification of the long-established gestural habits which consist, with “wet needle” syringes, in simply removing the needle-protecting cap by grasping and then pulling it. This imposed modification of their gestural habits has provided to be very badly accepted by users in the medical profession, and in practice, the “dry needle” injection devices currently represent a very small share of the market compared with that of the “wet needle” injection devices, despite the inherent disadvantages in the design of the latter.

The present invention aims to overcome this disadvantage of the “dry needle” injection devices of the third type described above, and has as its main object to provide an injection device which combines the advantages of these injection devices (sealing, safeguard against risks of possible leakage . . . ) and the activation of which for the purpose of an injection takes place naturally upon removal of the cap covering the base.

To this end, the invention relates to an injection device, comprising in combination:
a tubular receptacle provided with sealingly closing-off means at one of its longitudinal ends, the said sealingly closing-off means delimiting a chamber intended to be filled with a liquid, and comprising a liquid-delivery duct opening into the said chamber,
a needle-carrying base in which there is a duct for supplying liquid to the said needle, the said base being axially movable relative to the tubular receptacle, so as to be capable of displacement between a retracted position, called the closing-off position, in which the delivery duct is closed off, and an advanced position, called the injection position, in which it allows the liquid to flow out of the chamber to the needle through the supply duct of the said base,
a cap which is of a shape adapted to cover the base and is provided with means for detachable connection to the tubular receptacle,
mating axial-stop means formed on the base and the cap, and arranged so as to keep the said base in its retracted closing-off position when the latter is covered by the said cap connected to the tubular receptacle,
and axial-stop means arranged so as to limit the axial-displacement travel of the base, once the cap is separated from the tubular receptacle.

(It should be noted that, in this patent application, the term “needle-carrying base” is defined in a general way either as a base in which an injection needle is embedded, or a base consisting of a conical male locking connection.)

According to the invention, the injection device is therefore in an “inactivated” position, in its packaged configuration before use, in which the base is covered by the cap which ensures that the said base is kept in its closing-off position owing to the connection of this cap to the tubular receptacle.

In this configuration, the injection device therefore consists of an injection device of the “dry needle” type combining all the advantages of such injection devices. In addition, the activation of this injection device requires, for its part, simply the exerting of a pull on the cap for the purpose of detaching the latter from the tubular receptacle and thus releasing the base. This activation therefore results from a simple natural action identical to that performed at present for the purpose of releasing the needle of a “wet needle” injection device. This simple action leads, in fact, owing to the removal of the cap, to elimination of the axial stop keeping the base in its closing-off position, thus allowing the subsequent axial displacement of the said base to its injection position.

This axial displacement of the base may, furthermore, be caused solely just before the injection, under the effect of the pressure exerted by the liquid flowing out of the chamber of the receptacle following the actuation of the plunger of the injection device.

This axial displacement may also be obtained by providing friction surfaces between the base and the cap.

However, advantageously, for the purpose of bringing about this axial displacement of the base to its injection position, upon removal of the cap, the said base and the said cap comprise mating relative-translational-locking members arranged, on the one hand, so as to cooperate in the closing-off position of the base in which the latter is butting against the axial-stop means determining the said closing-off position, and, on the other hand, so as to allow relative axial displacement of the cap with respect to the base, in the injection position of the base in which the latter is butting against the axial-stop means determining the said injection position.

According to a first advantageous embodiment variant, the sealingly closing-off means and the base consist of two separate parts comprising means for relative translational locking of the said base with respect to the said sealingly closing-off means, provided, on the one hand, so as to allow displacement of the base from its closing-off position to its injection position, and, on the other hand, to translationally lock the said base with respect to the said sealingly closing-off means in the said injection position.

Thus, once the cap is removed, the base is automatically locked mechanically in its injection position, and therefore allows in particular a prior conventional drawing-up operation before injection, by pulling on the plunger rod, to be carried out without the risk of causing retraction of the said base which could prevent this drawing-up by closing off the delivery duct.

Moreover, according to this first embodiment variant and advantageously, the sealingly closing-off means comprise a closing-off stopper made of an impermeable elastic material having the shape of a hollow cylindrical body with a cross-section adapted to be inserted into the receptacle, the said closing-off stopper comprising a back wall for delimiting the chamber of the said receptacle, in which wall a passing-through delivery duct is formed. In addition, the injection device furthermore comprises:
a cover provided with an end-wall in which there is an orifice with a diameter adapted to permit the passage of the base, and with a cylindrical sleeve with a diameter adapted to fit together with the closing-off stopper, so that the said sleeve and stopper define a duct for guiding the said base,
means for relative translational locking of the closing-off stopper and the cover,
and means for relative translational locking of the closing-off stopper/cover assembly with respect to the receptacle.

According to this embodiment, the sealingly closing-off means are therefore dissociated into two separate elements of easy unitary manufacture:
a closing-off stopper made of a conventional material such as rubber, adapted to ensure the sealing between the sealingly closing-off means and the receptacle, and between the said closing-off means and the base,
and a cover produced, for example, from injection-moulded plastic and adapted to ensure that the closing-off stopper is locked and kept in place inside the receptacle, and to ensure, in collaboration with the said closing-off stopper, the guiding of the base during its displacement from its closing-off position to its injection position.

In addition, according to this embodiment, and advantageously, the means for translational locking of the base in its injection position comprise at least one radially deformable stop element arranged so as to be kept radially compressed inside the sleeve of the cover, in the closing-off position of the base and during displacement of the latter to its injection position, and to expand into an assigned locking area adapted to accommodate it in the injection position of the base.

Moreover, with regard to the detachable connection between the cap and the tubular receptacle, two distinct advantageous embodiments may be envisaged when the closing-off means and the base are formed of separate parts.

Thus, the cap and the cover may advantageously be produced in one piece and connected by detachable connection means consisting of a divisible connection extending between the said cap and the end wall of the said cover.

In contrast, the cap and the cover may also and advantageously consist of two separate parts comprising mating means for assembly by screwing.

In this second case, furthermore, the injection device advantageously comprises, for tamper-proof purposes, an annular crown produced in one piece with the cap and connected to the rear end of the latter by a divisible connection, the said annular crown being adapted to be accommodated inside the sleeve of the cover, in a translationally fixed position inside the said sleeve.

According to a second advantageous embodiment variant, the sealingly closing-off means and the base may also consist of a part, called the closing-off part, in one piece, provided with a section for sealingly closing off the tubular receptacle and adapted to delimit, with the said tubular receptacle, a sealed space with a capacity variable as a function of the longitudinal position of the said closing-off part, the supply duct and the delivery duct both being arranged so as to open into the sealed space in the advanced position of the closing-off part, and so that at least one of the said ducts is closed off by the tubular receptacle and isolated from the sealed space, in the retracted position of this closing-off part.

This solution, which tends to minimise the number of parts necessary for the manufacture of the injection device according to the invention, is therefore based on the creation, between the tubular receptacle and the closing-off part, of a sealed space having a delivery function allowing or not the injection of the liquid as a function of the longitudinal position of the said closing-off part.

Moreover, with regard to the tubular receptacle, the latter may advantageously consist of a simple cylindrical tube and consequently of an element which is very inexpensive to manufacture.

This tubular receptacle may also advantageously consist of a cartridge of the type of cartridge for anaesthetising liquid used in dentistry, comprising a neck having an outer enlargement at its end.

Such cartridges have, in fact, the advantage of being currently produced daily in very large numbers, and hence of a low cost price. Moreover, the dimensional characteristics of the neck of these cartridges are the same whatever the useful volume of this cartridge, and such a solution therefore makes it possible to produce a range of injection devices of different volumes, using a single design for parts forming the injection end-piece.

In the case of injection devices comprising such cartridges, furthermore and advantageously, the closing-off part has a shape adapted to slide and be guided inside the neck of the cartridge.

Moreover, and advantageously, an injection device according to the invention may be equipped at little expense with a case for protecting the needle after injection, comprising a tubular sheath adapted to slide along the tubular receptacle, the said sheath and the said tubular receptacle comprising relative-translational-locking means capable of determining an injection position in which the sheath is set back from the injection needle, and a protection position after use in which the sheath accommodates and masks the injection needle.

Further features, objects and advantages of the invention will emerge from the following detailed description with reference to the appended drawings which show by way of non-limiting examples seven embodiments of an injection device according to the invention, an alternative needle-protecting cap being able to be substituted for the needle-protecting cap with which the first two injection devices shown respectively in FIGS. 2-4 and 5-6 are equipped, and also two embodiments of injection devices according to the invention which are provided with a case for protecting the needle after injection. In these figures:

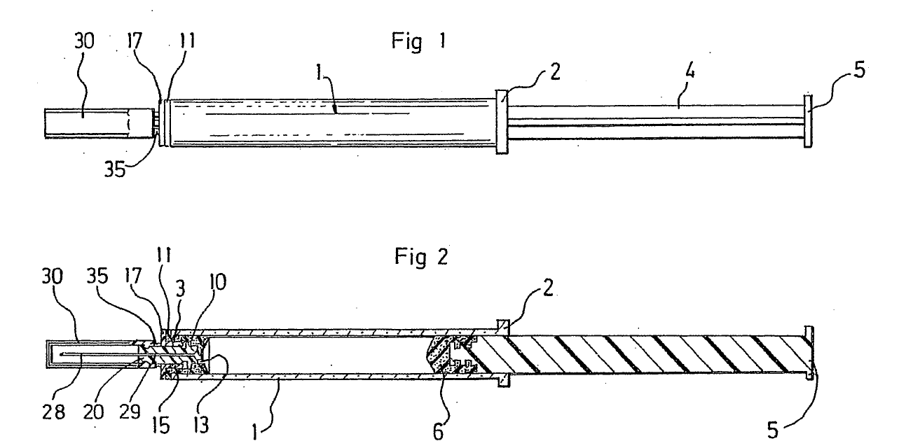

FIG. 1 is a general longitudinal view of a first injection device according to the invention, in its packaged configuration before use,

FIG. 2 is a longitudinal section of a first embodiment of the injection device as shown in FIG. 1, shown in its packaged configuration before use,

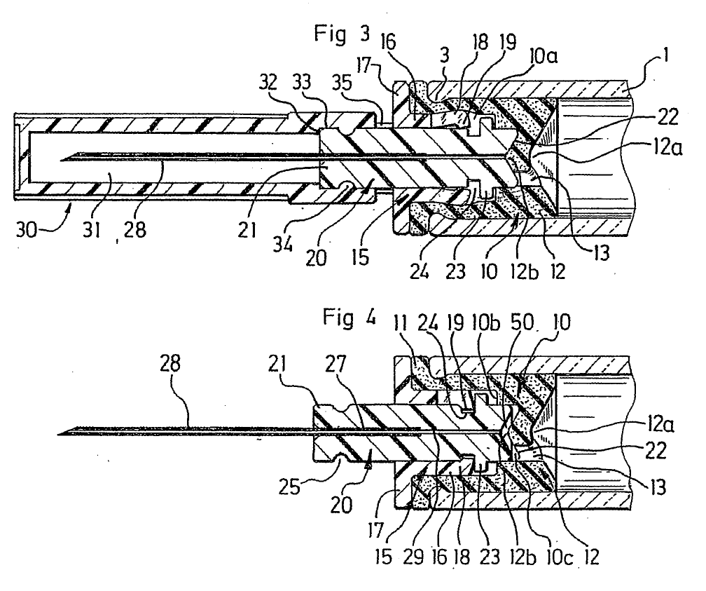

FIG. 3 is a partial longitudinal section on an enlarged scale of the injection device according to FIG. 2,

FIG. 4 is a partial longitudinal section on an enlarged scale showing the injection device according to FIG. 2, in its activated injection position, ready for use,

FIG. 5 is a partial longitudinal section on an enlarged scale of a second embodiment of the injection device as shown in FIG. 1, shown in its packaged configuration before use,

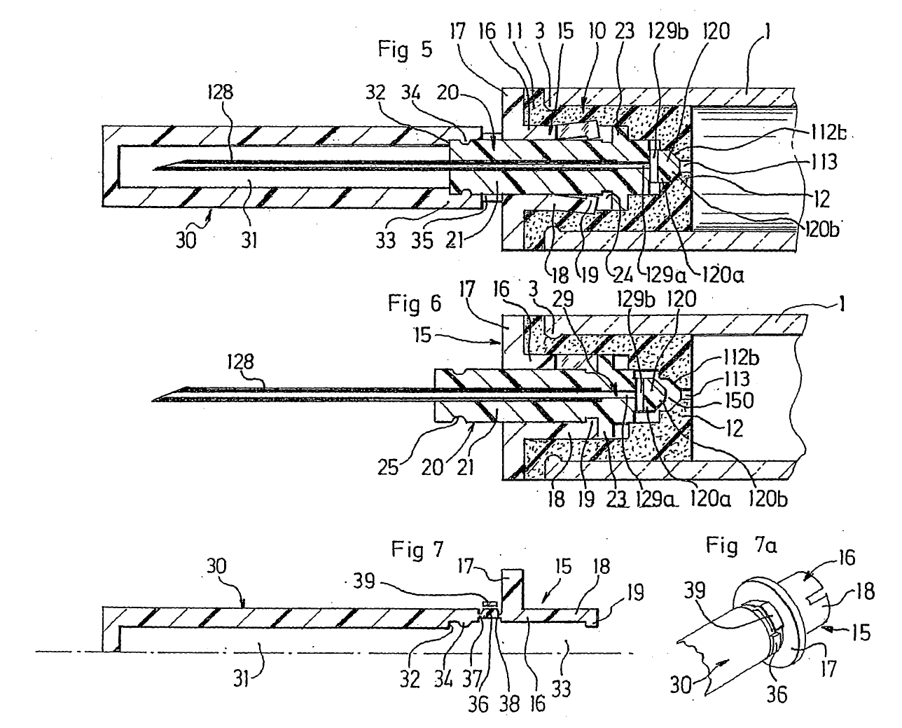

FIG. 6 is a partial longitudinal section on an enlarged scale showing this second embodiment in its activated injection position, ready for use,

FIG. 7 is a longitudinal section of an alternative needle-protecting cap which can be substituted for that shown in FIGS. 1, 2, 3, 5 with which the first two embodiments of injection devices according to the invention are equipped,

FIG. 7a is a partial perspective view of this needle-protecting cap,

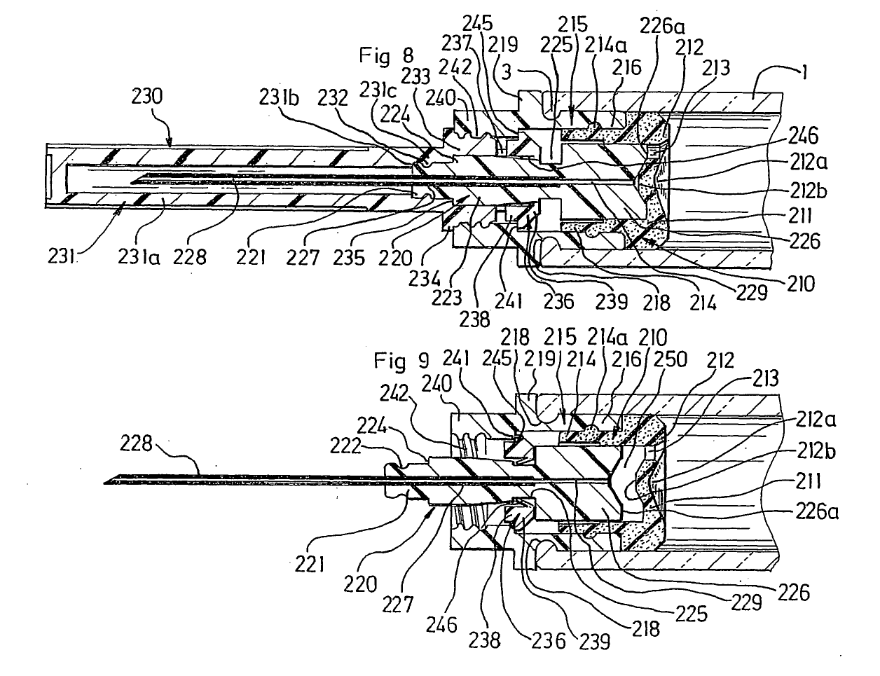

FIG. 8 is a partial longitudinal section of a third embodiment of the injection device according to the invention, shown in its packaged configuration before use,

FIG. 9 is a partial longitudinal section of this third embodiment of the injection device according to the invention, shown in its activated injection position, ready for use,

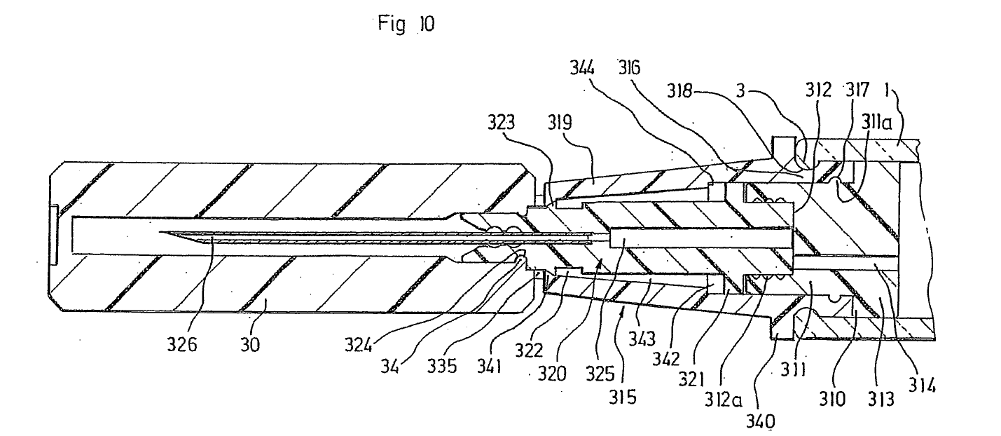

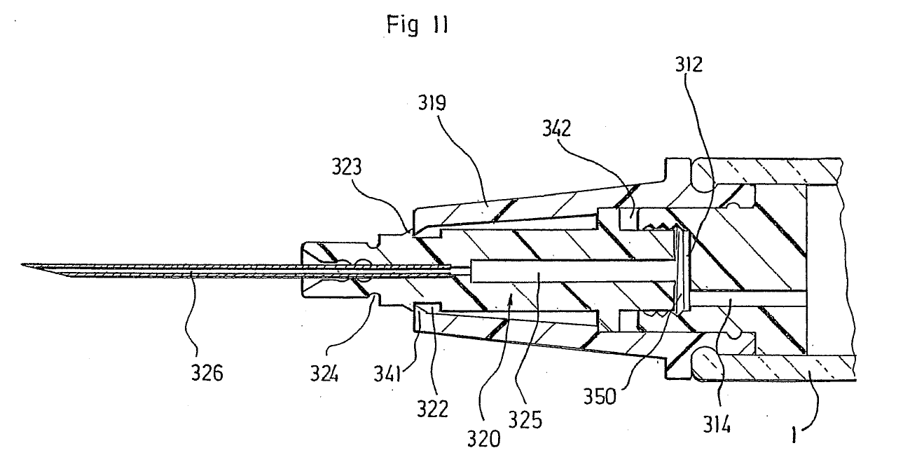

FIGS. 10 and 11 are partial longitudinal sections of a fourth embodiment of the injection device according to the invention, shown respectively in its packaged configuration before use, and in its activated position, ready for use,

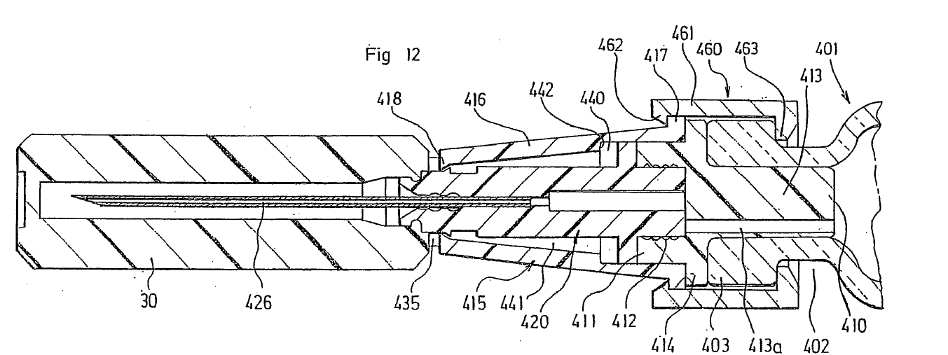

FIG. 12 is a partial longitudinal section of a fifth embodiment of the injection device according to the invention shown in its packaged configuration before use,

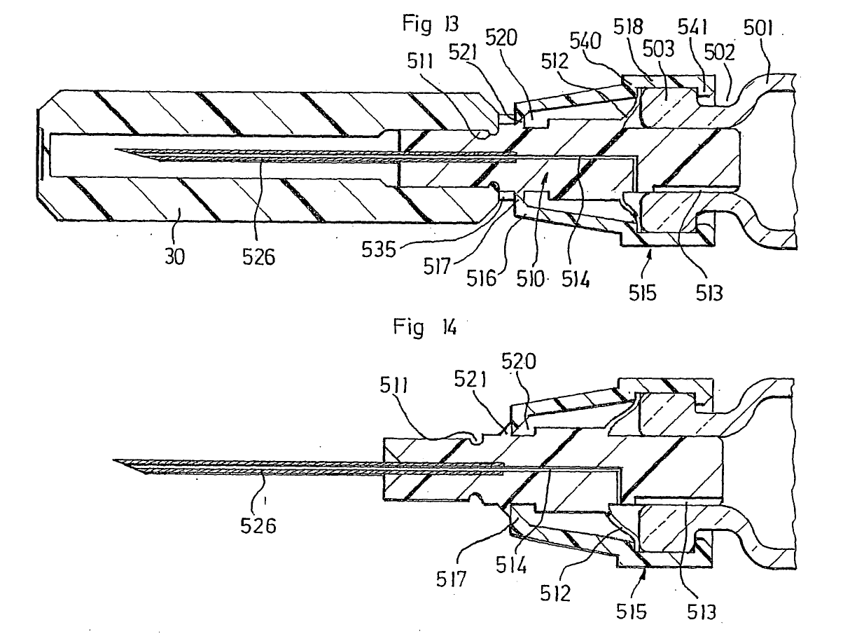

FIGS. 13 and 14 are partial longitudinal sections of a sixth embodiment of the injection device according to the invention, shown respectively in its packaged configuration before use, and in its activated position, ready for use,

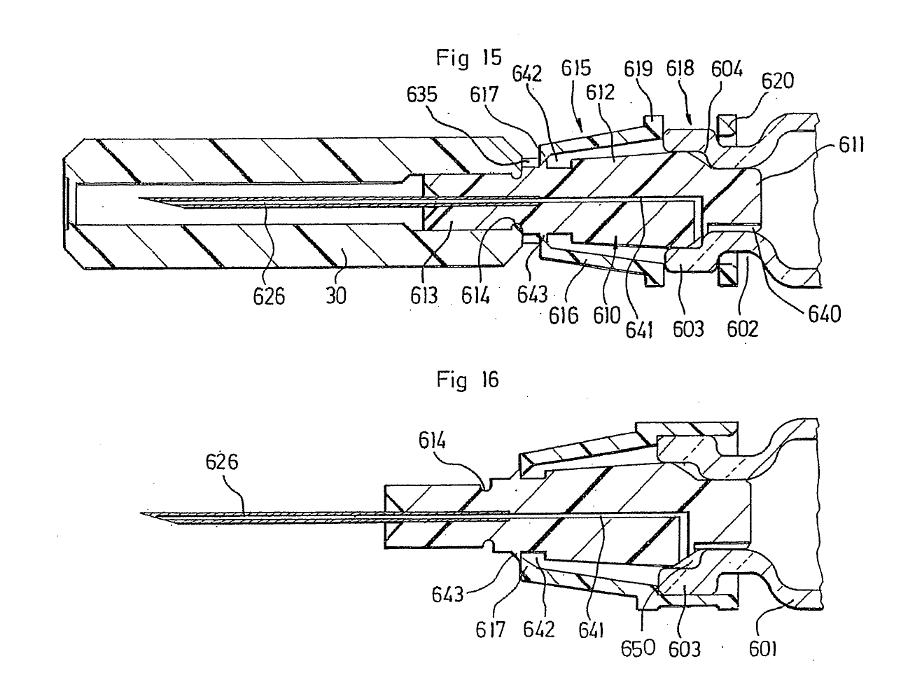

FIGS. 15 and 16 are partial longitudinal sections of a seventh embodiment of the injection device according to the invention, shown respectively in its packaged configuration before use, and in its activated position, ready for use,

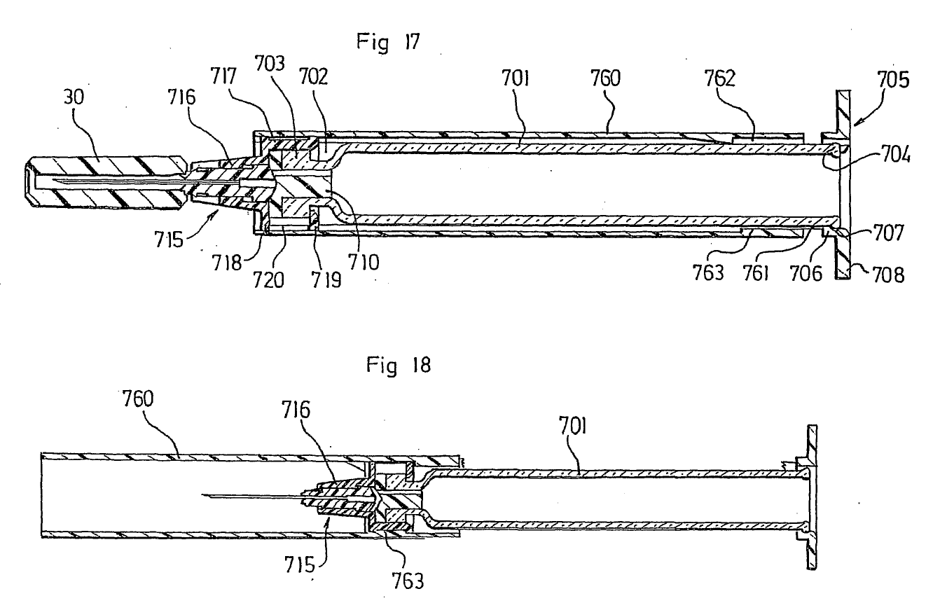

FIGS. 17 and 18 are longitudinal sections of the injection device according to the invention shown in FIGS. 13 and 14, which is further provided with a manual-release case for protecting the injection needle after use,

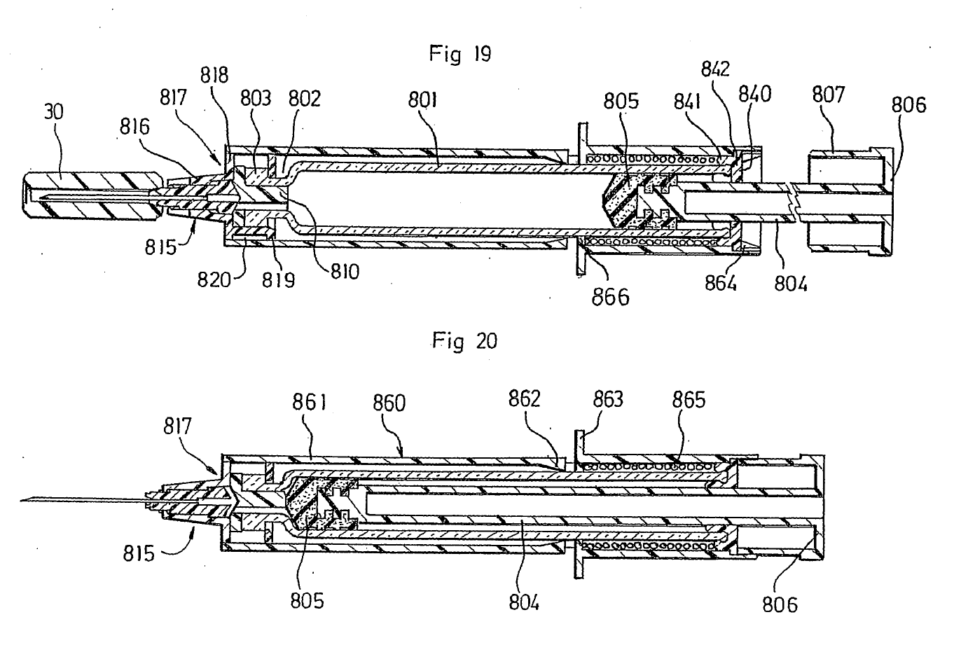

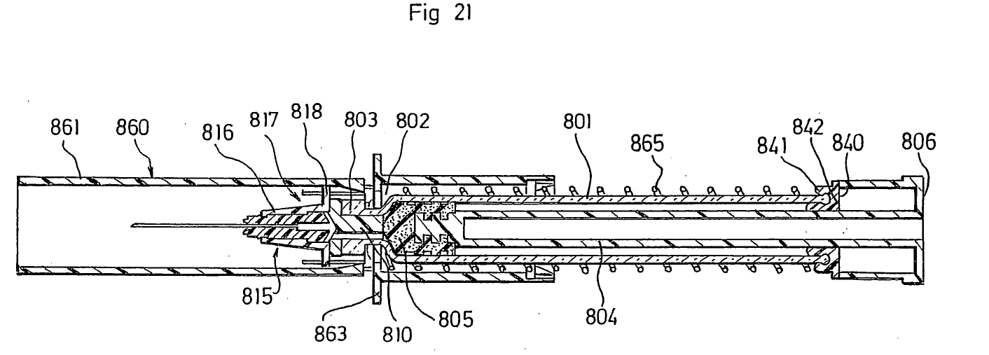

and FIGS. 19 to 21 are longitudinal sections of the injection device according to the invention shown in FIGS. 13 and 14, which is further provided with an automatic-release case for protecting the injection needle after use, and shown respectively in its packaged configuration before use (FIG. 19), at completion of the injection (FIG. 20), and in its protective position after use (FIG. 21).

The injection devices shown by way of non-limiting examples in FIGS. 1-6 and 8-16 are single-use injection devices, that is to say intended to be pre-filled with a dose of liquid to be injected. As shown in FIGS. 17 to 21, these injection devices may, furthermore, be equipped with a protective case intended to make it possible to protect the needle after injection and thus avoid any risk of being pricked with the contaminated needle.

As shown in FIG. 1, each of the injection devices according to the invention firstly comprises a syringe body 1, made of glass for example, a plunger 6 with a diameter adapted to slide sealingly inside the syringe body 1, and a plunger rod 4 secured by screwing to the plunger 6 at its front longitudinal end and provided with a finger-supporting pusher 5 at its rear end.

Firstly, with regard to the first four embodiments shown in FIGS. 1-6 and 10-11, the syringe body 1 consists of a simple cylindrical tube provided with an outer, finger-supporting flange 2 at one of its longitudinal ends, called the rear end. At its opposite end, called the front end, this cylindrical tube 1 furthermore has an inner annular rib 3 projecting inside the said tube.

Apart from these three elements, syringe body 1, plunger 6 and plunger rod 4, which are exactly the same for each of these four embodiments, each of the injection devices according to the invention shown in FIGS. 1-6 and 10-11 furthermore comprises four elements which fulfil a specific function common to the four embodiments and consist of:
a stopper, made of a conventional impermeable elastic material such as rubber, for sealingly closing off the front end of the syringe body 1,
a needle-carrying base movable axially relative to the syringe body 1 and therefore relative to the closing-off stopper,
a cover for locking and keeping the closing-off stopper in place,
and a needle-protecting cap adapted to cover the base and ensure the protection of the needle carried by the latter, in the packaged configuration before use of the injection device.

According to the embodiment shown in FIGS. 2 to 4, the closing-off stopper 10 is in the form of a hollow cylindrical body with an outer diameter mating with the inner diameter of the syringe body 1, adapted to be sealingly inserted inside the said syringe body. Internally, this closing-off stopper 10 is subdivided into two longitudinal cylindrical sections with different inner diameters separated by a radial shoulder 10b and consisting of a front section 10a and a rear section 10b with an inner diameter less than that of the front section 10a.

This closing-off stopper 10 furthermore comprises a front outer flange 11 for butting against the front longitudinal end of the syringe body 1.

This closing-off stopper 10 finally comprises a back wall 12 for delimiting, inside the syringe body 1, and with the plunger 6, the chamber containing the liquid to be injected. This back wall 12 has a frustoconical shape so as to form, at the rear, a concave seat 12a for supporting the plunger 6, and, at the front, a convex seat 12b for supporting the rear end-face of the base, described below.

The back wall 12 of the closing-off stopper 10 finally contains longitudinally a through-duct 13 for delivering the liquid contained in the chamber of the syringe body 1. This delivery duct 13 is furthermore eccentric relative to the longitudinal axis of the closing-off stopper 10, and arranged so as to open out at the periphery of the rear section 10b of the said stopper.

The cover 15 of this first embodiment consists, for its part, of a cylindrical sleeve 16 with an outer diameter mating with the inner diameter of the front section 10a of the closing-off stopper 10 and a length less than that of the said front section, adapted to be inserted in the latter so as to keep the said stopper clamped between the said sleeve and the syringe body 1, and translationally locked by deformation of the latter at the inner rib 3 of the said syringe body.

This cover 15 furthermore comprises an annular front end-wall 17 forming a flange on the periphery of the sleeve 16, for axially butting against the front flange 11 of the closing-off stopper 10.

Lastly, the cylindrical wall of the sleeve 16 of the cover 15 is divided up longitudinally, from its rear end, into longitudinal sectors such as 18, each delimited by two longitudinal slots, so as to form a radially deformable longitudinal tongue 18 in each case. In addition, a circumferential boss 19 is formed at the rear end of each tongue 18 so as to project into the sleeve 16 of the cover 15 in the absence of external stress exerted on the said tongue.

The base 20 of the first embodiment of the injection device according to the invention has, for its part, the general shape of a cylindrical shaft 21 with an outer diameter mating with the inner diameter of the rear section 10b of the closing-off stopper 10, adapted so that a sealed space 50 is delimited inside the said rear section by the back wall 12 of the closing-off stopper 10 and the rear end-face 22 of the said base, respectively.

This rear end-face 22 furthermore has a concave shape mating with the convex shape of the front seat 12b constituted by the back wall 12 of the closing-off stopper 10, and adapted to sealingly close off the delivery duct 13 opening out at the said seat.

The base 20 furthermore comprises an annular outer rib 23 arranged at a distance from the rear end-face 22 of this base 20 and adapted so that the said rib butts longitudinally against the radial inner shoulder 10b of the closing-off stopper 10, in a longitudinal position of the said base in which its rear end-face 22 rests against its supporting seat 12b.

The base 20 furthermore comprises a retaining annular peripheral groove 24, offset longitudinally towards the front relative to the annular rib 23 and adjoining the latter, the said groove being arranged so as to be positioned longitudinally:
on the one hand, set back to the rear relative to the bosses 19 of the sleeve 16 of the cover 15, in the retracted position of the base 20 in which the latter closes off the delivery duct 13 of the closing-off stopper 10,
and, on the other hand, so as to accommodate these bosses 19, in an advanced position of the base 20 in which the rear face 22 of the latter is spaced from the back wall 12 of the closing-off stopper 10, and a sealed space 50 is delimited between this rear face 22 and this back wall 12.

The base 20 also comprises a non-retaining annular peripheral groove 25 arranged longitudinally at a short distance from its front end, and intended for driving the said base from its position for closing off the delivery duct 13 to its position for freeing the liquid outlet orifice of the said duct, as explained below.

The base 20 lastly comprises an axial bore 27 which opens out at its front end-face and is intended in conventional fashion for embedding a single-point injection needle 28, and as a continuation of which there extends an axial duct 29 for supplying liquid to the said needle, which duct opens out at the rear face 22 of the said base.

The needle-protecting cap 30 consists, for its part, of a cap of conventional generally cylindrical outer shape. Internally, this cap 30 is subdivided into two longitudinal sections 31, 33 with different diameters, separated by a radial shoulder 32:
a rear section 33 with a short length relative to the total length of the cap 30, and with an inner diameter mating with the outer diameter of the base 20, and adapted to cover the latter,
and a front section 31 with an inner diameter less than that of the rear section 33, and adapted so that the shoulder 32 serves as an axial stop for the base 20.

Furthermore, the rear section 33 of the cap 30 has a non-retaining annular inner rib 34, positioned and with a shape adapted for seating in the annular groove 25 of the base 20, when this cap 30 covers the needle 28 and the said base.

Lastly, and according to the invention, the needle-protecting cap 30 is produced in one piece with the cover 15, for example by plastic injection moulding, and is secured to the front end-wall 17 of the said cover by a divisible connection of any type known per se, such as pins 35 shown in FIGS. 2 to 6.

As an alternative and as shown in FIGS. 7 and 7a, this divisible connection may also consist of an annular band 36 not closed on itself, which is connected to the cap 30 and the cover 15 by two divisible circular zones of weakness 37, 38 and is provided with a grasping tab 39 at its free end, for the purpose of tearing it.

The various elements of the injection device according to the invention which have been described above are assembled very easily by the following process:

Firstly, the base 20 is inserted into the cover 15 and the cap 30 until it butts against the inner shoulder 32 of the said cap, in which position:
the annular groove 25 of the base 20 accommodates the annular rib 34 of the cap 30,
the bosses 19 of the tongues 18 are in contact with the peripheral wall of the base 20, in front of the annular groove 24 of the said base, bringing about radial expansion of the said tongues.

The closing-off stopper 10 is then slipped at the rear onto the base 20 and the cover 15 until the flange 11 of the said stopper is brought into abutment at the rear against the front end-wall 17 of the said cover, in which position:
the base 20 is longitudinally locked between the inner shoulder 32 of the cap 30 and the inner shoulder 10b of the closing-off stopper 10,
the delivery duct 13 is closed off by the rear face 22 of the base 20.

The assembly thus pre-assembled is then simply forced into the syringe body 1 and thus forms an injection device of the “dry needle” type intended to be pre-filled, in which:
the injection needle 28 is isolated from the chamber containing the liquid to be injected by virtue of the delivery duct 13 being closed off by the base 20,
the sealing is ensured by the closing-off stopper 10 on the one hand at the peripheral faces of contact between the said stopper and the syringe body 1, and on the other hand at the peripheral faces of contact between this stopper 10 and the base 20.

For the purpose of its use, and in a manner essential according to the invention, the only action to be performed consists in removing the cap 30. During this removal, produced by the natural action performed for the purpose of removing the cap of a current conventional syringe, the divisible connection 35 is broken, then the base 20 is carried along with the cap 30 owing to the cooperation of the groove 25 and the rib 34, until the bosses 19 are seated in the annular groove 24 of the said base.

The cap 30 is then completely removed, while the base 20 is locked, without the possibility of retracting, in a position in which it delimits a sealed space 50 for communication between the delivery duct 13 and the supply duct 29 of the needle 28.

It should be noted that, according to the alternative shown in FIGS. 7 and 7a, the process is exactly the same once the preliminary step of tearing the annular band 36 has been performed.

The injection device shown in FIGS. 5 and 6 consists of elements which, overall, are similar to those of the injection device described above.

Thus, in particular, the syringe body 1 and the cover 15/needle-protecting cap 30 assembly are exactly the same as those described above and will not therefore be described again in detail.

The base 20 and the closing-off stopper 10 are, for their part, generally identical to those described above, the only differences involving the design of the “valve system” formed by the rear section of the base 20 and the back wall 12 of the closing-off stopper 10.

Only this “valve system” therefore is described in detail below with new reference numerals, it being understood that the above description should be referred to for the other elements which constitute the base 20 and the closing-off stopper 10.

According to this embodiment, and firstly, the front face of the back wall 12 of the closing-off stopper 10 is hollowed out with an axial seat 112b of generally cylindro-ogival shape into which an axial delivery duct 113 opens.

The base 20 of this injection device comprises, for its part, as before, a cylindrical shaft 16 with an outer diameter mating with the inner diameter of the rear section 10b of the closing-off stopper 10, and as a rear continuation of the said shaft, an obturator 120 of generally cylindro-ogival shape mating with that of the seat 112b.

This obturator 120 furthermore has a front cylindrical section 120a with a length greater than that of the cylindrical section of the seat 112b and adapted so that, in the position in which the said seat is closed off by the said obturator, the front portion of the front section 120a of this obturator 120 extends outside the seat 112, into a sealed chamber 150 delimited by the back wall 112 of the closing-off stopper 10 and the rear end-face 22 of the base 20.

According to this embodiment, furthermore, the supply duct 129 has the shape of a T consisting:
of a longitudinal axial leg 129a extending as a rear continuation of the axial bore 27 in which the injection needle 28 is embedded,
of a radial rear through-leg 129b arranged in the front portion of the front section 120a of the obturator 120, so as to open, at each of its two ends, into the sealed chamber 150.

According to this embodiment, in the retracted position of the base 20 shown in FIG. 5, the obturator 120 closes off the delivery duct 113, and the sealed chamber 150 is isolated from the chamber of the receptacle containing the liquid to be injected.

In contrast, as shown in FIG. 6, after removal of the needle-protecting cap 30, and consequently in the advanced injection position of the base 20, the supply duct 129 and the delivery duct 113 both open out into the chamber 150, allowing the liquid to flow out to the injection needle 28.

The injection device shown in FIGS. 8 and 9 differs mainly from those described above in that the cover and the needle-protecting cap consist not of a part produced in one piece but of two separate parts adapted to be assembled by screwing.

As before, the same reference numerals are used below to denote elements of similar structure, common to the three embodiments.

According to this third embodiment, and firstly, the closing-off stopper 210 is composed of a rear portion which is in the form of a cylindrical cup 211 with an outer diameter mating with the inner diameter of the syringe body 1 and is adapted to be sealingly inserted into the said syringe body.

This cup 211 furthermore comprises a back wall 212 similar to the back wall 12 of the closing-off stopper 10 described above with reference to FIGS. 2 to 4, that is to say which forms a concave rear seat 212a, a convex front seat 212b, and in which there is arranged a delivery duct 213 offset from the longitudinal axis of this cup.

The closing-off stopper 210 furthermore comprises a front portion 214 consisting of a cylindrical sleeve with an inner diameter substantially similar to that of the said cup, and with an outer diameter adapted to form a longitudinal annular space between the said sleeve and the inner face of the syringe body 1.

This cylindrical sleeve 214 finally comprises an annular outer rib 214a arranged to project from its peripheral wall, substantially halfway along the said sleeve.

The cover 215 of this third embodiment comprises, for its part, a cylindrical rear sleeve 216 with a cross-section adapted to be inserted into the annular space formed between the syringe body 1 and the sleeve 214 of the closing-off stopper 210.

At its peripheral inner face, this sleeve 216 has an annular inner groove 217 arranged substantially halfway along the said sleeve and adapted to accommodate the annular rib 214a of the sleeve 214 of the closing-off stopper 210, and thus ensure relative translational locking of the said cover and closing-off stopper.

In addition, this sleeve 216 has an annular outer groove 218 arranged at the front end of the said sleeve and adapted to accommodate the annular inner rib 3 of the syringe body 1 and thus ensure that the closing-off stopper 210/cover 215 assembly is locked and kept in place inside the said syringe body.

The cover 215 furthermore comprises an annular front end-wall 219 forming an outer flange on the periphery of the sleeve 216, for butting against the front end of the syringe body 1.

This cover 215 finally comprises a cylindrical front sleeve 240 centred on the axis of the annular front end-wall 219 of the said cover and projecting forwards from the front face of the said end-wall.

This front sleeve 240 has an outer diameter matching that of the rear sleeve 216, and an inner diameter less than the identical inner diameters of the said rear sleeve and of the orifice of the end-wall 219, and adapted to form a radial inner shoulder 241 at its junction with the said end-wall.

Finally, this front sleeve 240 has, starting from its front end, a longitudinal internally threaded section 242.

The base 220 of this third embodiment consists, for its part, of a part of generally cylindrical shape subdivided into three longitudinal sections:
a front section 221 with a smaller diameter, in the peripheral wall of which there is arranged, at a short distance from its front end, a non-retaining annular outer groove 222,
an intermediate section 223 with a diameter greater than that of the front section 221, adapted to form a radial outer shoulder 224 at its junction with the said front section. In addition, an annular outer groove of retaining shape 225 is arranged in the peripheral wall of this intermediate section, at its rear end section,
and a rear section 226 with an outer diameter mating with the inner diameter of the sleeve 214 of the closing-off stopper 210, adapted to be sealingly inserted into the said sleeve, and to define, inside this sleeve 214, a sealed space 250 delimited by the back wall 212 of the closing-off stopper 210, and the rear end-face 226a of the said rear section. This rear end-face 226a of this rear section 226 furthermore has a concave shape mating with the convex shape of the front seat 212b of the back wall 212 of the closing-off stopper 210, and adapted to sealingly close off the delivery duct 213 opening out at the said seat.

The base 220 lastly comprises an axial bore 227 opening out at its front end-face and extending over a length substantially equal to the combined length of the front section 221 and intermediate section 223, the said bore being conventionally intended for embedding a single-point injection needle 228. In addition, this base 220 conventionally contains, as a continuation of this axial bore 227, an axial duct 229 for supplying liquid to the injection needle 228 and opening out at the rear end-face 226a of the said base.

The needle-protecting cap 230 of this third embodiment comprises, for its part, firstly, a front portion 231 having the generally cylindrical shape of a needle-protecting cap of conventional type.

Internally, this front portion 231 is subdivided into two longitudinal sections 231a, 231c of generally cylindrical shapes and with different inner diameters, separated by a radial inner shoulder 231b: a front section 231a extending over most of the length of this front portion 231, and a rear section 231c of short length compared with that of the front section 231a, and with a diameter greater than that of the latter.

Furthermore, a non-retaining annular inner rib 232 is arranged to project inside the rear section 231c, at a short distance from the shoulder 231b.

The needle-protecting cap 230 furthermore comprises, as a rear continuation of its front portion 231, an externally threaded cylindrical sleeve 233, with a diameter and threading adapted to be screwed into the internally threaded sleeve 240 of the cover 215.

In addition, this cylindrical sleeve 233 has an outer flange 234 for frontally butting against the front end-face of the internally threaded sleeve 240, when the said cylindrical sleeve is inserted longitudinally and screwed into the said internally threaded sleeve.

This cylindrical sleeve 233 furthermore has an inner diameter mating with the outer diameter of the intermediate section 223 of the base 220, and greater than that of the rear section 231c of the front portion 231 of the cap 230, adapted to delimit a radial inner shoulder 235 at the junction between the said rear section 231c and rear sleeve 233.

The needle-protecting cap 230 according to this embodiment is, moreover, continued at the rear by an annular ring 236 of generally cylindrical shape, connected to the rear end-face of the externally threaded cylindrical sleeve 233 by a divisible connection 237 of the pin type.

This annular ring 236 has an inner diameter mating with the outer diameter of the intermediate section 233 of the base 220, adapted to make it possible to insert the said base into the said ring and the cylindrical sleeve 233.

This annular ring 236 is, furthermore, subdivided longitudinally into two sectors:
a front cylindrical sector 238 with a cross-section adapted to slide inside the cylindrical sleeve 233, along the intermediate section 223 of the base 220,
a rear sector 239 of frustoconical shape, having at its junction with the front sector 238 a diameter adapted to form an annular shoulder 245 radial relative to the peripheral face of the said front sector, for frontally butting against the radial inner shoulder 241 of the cover 215.

Finally, this annular ring 236 comprises at least one radially elastic tongue 246 projecting inside the said ring, so as to be kept compressed against the inner peripheral face of this ring 236, by the intermediate section of the base 220, in the retracted position of the said base.

This tongue 246 is, furthermore, attached to the ring 236 at the rear end of the latter, so as to be able to expand radially and be seated in the retaining groove 225 of the said base, in the advanced injection position of the latter, and thus prevent any subsequent retraction of this base 220.

During the use of this injection device, the removal of the cap 230 by unscrewing brings about, firstly, a rupture of the divisible connection 237, which therefore constitutes a guarantee that the injection device is tamper-proof.

The injection device shown in FIGS. 10 and 11 is similar in design to those described with reference to FIGS. 3-4 and 5-6 and therefore comprises a needle-protecting cap 30 and a cover 315 produced in one piece and connected by a divisible connection 335 consisting, for example, of divisible pins.

According to this embodiment, and firstly, the stopper 310 is adapted to extend partially as a front continuation of the syringe body 1, so that the sealed space 350 for the outflow of the liquid to be injected is arranged outside and in front of the said syringe body.

This stopper 310 has longitudinally a T-shaped cross-section formed, on the one hand, of a front cylindrical body 311 with an outer diameter less than the inner diameter of the syringe body 1, in the front end-wall of which is arranged a cylindrical cavity 312 forming a liquid-outflow chamber 350, and, on the other hand, of a rear cylindrical sealing flange 313 with an outer diameter mating with the inner diameter of the syringe body 1.

This stopper 310 furthermore comprises annular decompression grooves 312a arranged in the outflow chamber 350. It also comprises an annular outer rib 311a arranged on the cylindrical body 311 at a short distance from the flange 313.

As before, this stopper 310 finally contains a delivery duct 314 offset from the longitudinal axis of the said stopper and adapted to open into the back of the cylindrical cavity 312.

The cover 315 of this injection device has, for its part, a shape adapted to form a “frustoconical nib” as a front continuation of the syringe body 1.

This cover 315 firstly comprises a rear section 316 of short length forming a cylindrical sleeve adapted to be inserted into the annular space formed between the syringe body 1 and the cylindrical body 311 of the stopper 310.

This rear section 316 furthermore comprises, as before, an annular inner groove 317, and an annular outer groove 318 adapted to cooperate respectively with the annular outer rib 311a of the stopper 310 and the annular inner rib 3 of the syringe body 1, so as to ensure relative translational locking of the syringe body 1/stopper 310/cover 315 assembly, in a position in which the cavity 312 of the stopper 310 extends outside and in front of the syringe body 1.

The cover 315 furthermore comprises a front section 319 in the form of a frustoconical sleeve adapted to extend as a continuation of the syringe body 1 and separated from the rear section 316 by an outer flange 340 for butting against the front end of the syringe body 1. This front section 319 comprises an annular inner rib 341 arranged at the front end of this front section, and having a sawtooth-shaped cross-section, the ramp-forming inclined face of which is oriented towards inside of the said front section.

The longitudinal through-bore delimited by the peripheral wall of this cover 315 comprises, for its part, a cylindrical rear section 342 with an inner diameter mating with the outer diameter of the cylindrical body 311 of the stopper and with a length greater than that of the said cylindrical body, and adapted to form a cylindrical space in front of the said stopper seated and locked in the said cover.

This bore furthermore comprises, as a longitudinal continuation of the rear section 342, a frustoconical front section 343 with a maximum diameter less than that of the said rear section, so as to form a radial shoulder 344 at the junction of these two sections.

The base 320 of this injection device is similar to that of the injection devices described with reference to FIGS. 3 to 6 and is therefore in the general form of a cylindrical shaft with an outer diameter adapted to penetrate into the cavity 312 of the stopper 310 so as to delimit a sealed space inside the said cavity.

This base 320 furthermore comprises an annular outer rib 321 arranged and with an inner diameter adapted for seating in the cylindrical rear section 342 of the cover 315 and to allow longitudinal displacement of the said base relative to the said cover between:
a retracted position in which the rib 321 is adjacent to the stopper 310,
and an advanced position in which this rib 321 is butting against the radial shoulder 344 of the cover 315.

The base 320 moreover comprises an annular outer groove 322 of retaining shape delimited by a front edge 323 of sawtooth-shaped cross-section complementary to that of the inner rib 341 of the cover 315, so as to allow the penetration of the said rib 341 into the said groove 322 upon displacement of the base 320 towards its advanced position, and then to prevent any return of the said base towards its retracted position.

This base 320 furthermore comprises a non-retaining annular groove 324 adapted to cooperate with the annular inner rib 34 of the needle-protecting cap 30, so as to ensure that the said base is kept in a retracted position in which it closes off the delivery duct 314 of the stopper 310, and in which it extends between the back of the cavity 312 and the radial shoulder formed in the cap 30.

Lastly, as before, the base 320 contains longitudinally an axial duct 325 for supplying liquid to be injected to the injection needle 326 embedded in the said base.

The injection device shown in FIG. 12 differs mainly from that described above with reference to FIGS. 10 and 11 in that the receptacle 401 of this injection device consists, in place of the cylindrical tube 1, of a cartridge 401 or “carpule” of the type conventionally used in dentistry for the purpose of packaging and injecting anaesthetising liquids.

This cartridge 401 therefore comprises, in conventional fashion, a cylindrical tube having, at one of its ends, a narrowing delimiting a cylindrical neck 402 provided with a cylindrical outer enlargement 403 at its end, formed of an increased thickness of the peripheral wall of the said neck.

The stopper 410 of this injection device is similar to the stopper 310 described above, and comprises a cylindrical front body 411 adapted to extend as a continuation of the cartridge 401, and in the front wall of which is arranged a cylindrical cavity 412, a cylindrical rear body 413 with an outer diameter adapted to penetrate sealingly into the neck 402 of the cartridge 401, and an intermediate outer flange 414 formed at the junction between the front body 411 and rear body 413, and with an outer diameter substantially matching that of the enlargement 403 of the said cartridge and adapted so that the said flange butts against the said enlargement.

As before, this stopper 410 finally contains an offset delivery duct 413a arranged in the rear body 413 so as to open into the back of the cavity 412.

The cover 415 of this injection device is, as before, shaped so as to form a “frustoconical nib” as a continuation of the neck 402 of the cartridge 401.

This cover 415 consists of a frustoconical end-piece 416 adapted to cover the front body 411 of the stopper 410 and extend as a continuation of the latter, the said end-piece comprising, at its rear end, an outer flange 417 for butting against the intermediate flange 414 of the stopper 410, and, at its front end, an annular inner rib 418 of sawtooth-shaped cross-section.

The longitudinal through-bore delimited by the peripheral wall of this cover 415 comprises, for its part, a cylindrical rear section 440 with an inner diameter mating with the outer diameter of the front body 411 of the stopper 410, and with a length greater than that of the said front body, adapted to form a cylindrical space in front of the said stopper.

This bore furthermore comprises a frustoconical front section 441 with a diameter less than that of the rear section 440, adapted to form a radial shoulder 442 at the junction of these sections.

The base 420 of this injection device is, for its part, identical to the base 320 described above, and is adapted to be capable of displacement longitudinally inside the cover 415 between:
a retracted position for closing off the delivery duct 413a,
and an advanced position for the outflow of the liquid into the cavity 412 of the injection stopper, and then to the needle 426.

This injection device finally comprises a ring 460 for securing the cover 415 to the cartridge 401, consisting of a cylindrical ring 461 made of a material such as aluminium, split longitudinally so as to be capable of radial elastic deformation.

This ring 461, provided with an annular inner rib 462, 463 at each of its ends, has an inner diameter mating with the outer diameter of the enlargement 403 of the neck 402 of the cartridge 401, and a length adapted to keep the flanges of the cover 415 and of the base 420 up against the enlargement 403 of the cartridge 401.

As before, the injection device therefore comprises a base 420 in which is embedded an injection needle 426 protected by a cap 30 connected to the cover 415 by a divisible connection 435, the cap 30/cover 415, stopper 410, cartridge 401, ring 460 assembly:
forming an injection device constructed to ensure that the base 420 is kept in its retracted position for closing off the delivery duct 413a,
being adapted to bring about the displacement of the base 420 towards its advanced position, then its locking in this advanced position, upon removal of the cap 30.

The injection device shown in FIGS. 13 and 14 comprises, like the preceding one, a syringe body consisting of a cartridge 501, and has mainly the particular feature of comprising a stopper and a base which are formed in one and the same piece, and also a cover and a ring which are likewise formed in one and the same piece.

Firstly, the stopper/base 510 is formed of a cylindrical shaft with a diameter adapted to penetrate into the neck 502 of the cartridge 501, and with a length adapted to extend substantially over two-thirds of its length as a continuation of the said cartridge.

Like the bases 320 and 420 shown in FIGS. 10 to 12, this stopper/base 50 furthermore has a non-retaining annular outer groove 511, for connection to the cap 30, and also a retaining outer groove 520 delimited at the front by an edge 521 of sawtooth-shaped cross-section, for locking the said stopper/base in its advanced injection position.

Specifically, this stopper/base 510 comprises a flexible sealing skirt 512 arranged so as to extend in front of the neck 502 of the cartridge 501 when the said stopper/base is introduced into the said neck.

In addition, this stopper/base 510 comprises at least one channel 513 arranged, starting from its rear end, over a length adapted to be entirely accommodated in the neck 502 of the cartridge 501, in the retracted position of the stopper/base, and to open to the outside of the said neck in the advanced position of the said stopper/base.

This stopper/base 510 finally comprises a duct 514 for supplying the injection needle 526 having an axial leg as a rear continuation of the said needle, and also at least one radial emerging leg arranged so that its outlet orifice is positioned longitudinally between the skirt 512 and the end of the channel 513.

As mentioned above, the cover and the ring of this injection device consist of an assembly part 515 in one piece connected to the needle-protecting cap 30 by a divisible connection 535, and of an overall shape similar to that of the cover 415 and of the ring 460 described above, in the assembled state of these latter, the said assembly part therefore comprising:
a frustoconical front section 516 provided at its front end with an inner rib 517 constructed for seating in the retaining groove 520 of the stopper/base 510, in the advanced position of the latter,
a cylindrical rear section 518 delimited longitudinally by two inner ribs 540, 541, one of which is an intermediate rib for joining the front section 516 and rear section 518 and is arranged so as to keep the peripheral edge of the skirt 512 sealingly clamped against the front end-face of the enlargement 503 of the neck 502 of the cartridge 501.

According to this embodiment, in the packaged configuration of the injection device, the stopper/base 510 is kept in its retracted position in which the delivery duct 513 is closed off by the peripheral inner face of the neck 502 of the cartridge 501 and therefore does not allow any outflow of liquid (FIG. 13).

In contrast, removal of the cap brings about irreversible longitudinal displacement of the stopper/base 510 towards its advanced position in which the delivery duct 513 opens into the chamber sealingly delimited by the skirt 512, and into which there also opens the supply duct 514, into which the liquid is therefore delivered during injection.

FIGS. 15 and 16 show another embodiment of the injection device according to the invention comprising, like the preceding one, a stopper/base assembly in one piece. According to this embodiment, furthermore, the neck 602 of the cartridge 601 and the stopper/base 610 are shaped so as to have complementary faces adapted to ensure directly the sealing of the chamber of the cartridge 601 containing the liquid to be injected in the retracted position of the said stopper/base, and to delimit a sealed space 650 for the outflow of this liquid, in the advanced position of this stopper/base 610.

For this purpose, and firstly, the peripheral wall of the neck 602 has a constant thickness so that the enlargement 603 delimits internally a seat 604 of substantially retaining shape and with a diameter greater than that of the neck 602.

The stopper/base 610 comprises, for its part, a cylindrical rear section 611 with a diameter adapted to penetrate sealingly into the neck 602 of the cartridge 601, and along which at least one channel 640 is arranged.

This stopper/base 610 furthermore comprises an intermediate section 612 of slightly conical shape complementary to that of the seat 604 of the cartridge 601 and adapted to fit the shape of this seat 604, in its retracted position, and to delimit a sealed space 650 with the said seat, in its advanced position.

This stopper/base 610 also comprises a front section 613 for cooperation with the cap 30, and in which the injection needle 626 is embedded.

As before, this stopper/base 610 moreover comprises a non-retaining outer groove 614 for connection to the cap 30, and also a retaining outer groove 642 delimited by a front edge 643 of sawtooth-shaped cross-section, for locking the latter in its advanced position.

This stopper/base 610 finally comprises a supply duct 641 formed of an axial leg as a rear continuation of the injection needle 626, and of at least one radial leg arranged so as to open, downstream of the end of the channel(s) 640, into the sealed space 650 formed in the advanced position of the stopper/base.

FIGS. 17-18 and 19-21 show, for their part, respectively an injection device according to the invention corresponding to any one of the embodiments described above, equipped, furthermore, with a protective case adapted to mask the needle after injection.

By way of example, the injection device shown in these figures comprises a syringe body consisting of a cartridge 701 (801) provided with a neck 702 (802) on which is shaped an annular enlargement 703 (803).

This injection device also comprises, according to the invention, a stopper 710 (810), a base 720 (820), a cover 715 (815) connected to a needle-protecting cap 30 by a divisible connection, and also a ring 717 (817), in the example in one piece with the cover 715 (815), for translationally securing all of these parts to the syringe body 701 (801).

Furthermore, specifically, according to this type of embodiment with protective case, the two ribs 718, 719 (818, 819) described above and delimiting the ring 717 (817) have a radial thickness adapted, furthermore, to form in each case an annular crown on the periphery of the said ring, and to delimit an annular receptacle 720 (820) between them.

Firstly, FIGS. 17 and 18 show an injection device according to the invention provided with a protective case for manual release after injection.

This injection device comprises a finger-supporting flange 705 provided with a sleeve 706 for fitting onto the end of the cartridge 701 opposite the neck 702, and provided with an annular inner groove 707 constructed to catch on an annular outer groove 704 arranged on the said cartridge.

The protective case 760 consists, for its part, of a cylindrical sheath connected at one of its ends to the finger-supporting flange 705 by a divisible connection 761, and with an inner diameter mating with the diameter of the crowns 718, 719, and adapted so that the said crowns form supporting surfaces for guiding the said sheath as it slides.

For the purpose of its irreversible translational locking in its advanced position for protecting the needle after injection, this sheath 760 furthermore comprises, arranged inside the said sheath, a rib 763 (in the form of a stud or studs or annular) adapted for positioning in the outer receptacle 720 of the ring 717. Similarly, at least one groove 762 adapted to longitudinally cover the crowns 718, 719 of the ring 717 is formed inside the said sheath (of course, these locking members are conventionally designed so as to form ramps, and to possess elasticity, enabling their relative clearance).

According to this embodiment, and at completion of the injection, the protection of the needle is obtained by pulling on the sheath 760 so as to bring about the rupture of the divisible connection, then the sliding of the said sheath as far as its advanced position in which it is locked irreversibly relative to the syringe body 701.

FIGS. 19 to 21 show, for their part, an injection device provided with a protective case for automatic release at completion of the injection.

As in the preceding example, the syringe body consists of a cartridge 801 provided with a neck 802 on which an enlargement 803 is formed. In addition, a plunger 805 secured to the end of a plunger rod 804 (not shown in the preceding example) is adapted to slide inside this cartridge 801 for the purpose of injecting the liquid contained in the latter.

This plunger rod 804 furthermore comprises a pusher member 806 in the form of a cylindrical cup having a finger-supporting rear end-wall, on the periphery of which a cylindrical sleeve 807 extends perpendicularly.

The syringe body 801 is furthermore closed off at its end opposite the neck 802 by a circular closing-off plate 840, from which there projects a cylindrical connector 841 for catching on the said syringe body.

According to this embodiment, the protective case 860 consists of a sheath 861 provided at one of its ends with radially deformable catching hooks 864 adapted to hook onto the rear face of the closing-off plate 840 and to delimit, with the rear end 842 of the said sheath, an annular inner recess for translationally locking this sheath 861 relative to the said plate and therefore relative to the syringe body 801.

This sheath 861 furthermore comprises an annular inner rib 866 arranged at a distance from its rear end 842 and adapted to delimit, around the syringe body 801, and with the closing-off plate 840, an annular space accommodating a spring 865 kept in its compressed state.

This sheath 861 also comprises a finger-supporting outer collar 863, in the example extending in the same radial plane as the rib 866, and also substantially in front of the said rib, an inner rib 862 (in the form of a stud or studs or annular) adapted for seating in the outer receptacle 820 of the ring 817, in the advanced position of the sheath 861 in which the latter protects the needle after injection.

According to this embodiment, and as shown in FIG. 20, when the plunger 805 reaches the end of its travel, the sleeve 807 comes into contact with the catch hooks 864 of the sheath 861 and causes radial deformation of these latter which frees the hooking of the said sheath on the closing-off plate 840.

Under the effect of the relaxation of the spring 865 bearing on the inner rib 866 of the sheath 861 and the closing-off plate 840, the sheath 861 is then automatically caused to slide along the syringe body 801 until it reaches its advanced position for protecting the needle, in which it is irreversibly locked by virtue of the penetration of the rib 862 into the outer receptacle 820 of the ring 817 (FIG. 21).

It should be noted, furthermore, that in this advanced position, the rib 866 butts against the rear face or the rear rib 819 of the ring 817, also preventing the sheath 861 from continuing its forward displacement.

1-13. (canceled)

14. Single-use injection device, wherein it comprises in combination:
a tubular receptacle provided with sealingly closing-off means at one of its longitudinal ends, the said sealingly closing-off means delimiting a chamber intended to be filled with a liquid, and comprising a liquid-delivery duct opening into the said chamber,
a needle-carrying base in which there is a duct for supplying liquid to the said needle, the said base being axially movable relative to the tubular receptacle, so as to be capable of displacement between a retracted position, called the closing-off position, in which the delivery duct is closed off, and an advanced position, called the injection position, in which it allows the liquid to flow out of the chamber to the needle through the supply duct of the said base,
a cap which is of a shape adapted to cover the base and is provided with means for detachable connection to the tubular receptacle,
mating axial-stop means formed on the base and the cap, and arranged so as to keep the said base in its closing-off position when the latter is covered by the said cap connected to the tubular receptacle,
and axial-stop means arranged so as to limit the axial-displacement travel of the base, once the cap is separated from the tubular receptacle.

15. Injection device as claimed in claim 14, wherein the base and the cap comprise mating relative-translational-locking members arranged, on the one hand, so as to cooperate in the closing-off position of the base in which the latter is butting against the axial-stop means determining the said closing-off position, and, on the other hand, so as to allow relative axial displacement of the cap with respect to the base, in the injection position of the base in which the latter is butting against the axial-stop means determining the said injection position.

16. Injection device as claimed in claim 15, wherein the sealingly closing-off means and the base consist of two separate parts comprising means for relative translational locking of the said base with respect to the said sealingly closing-off means, provided, on the one hand, so as to allow displacement of the base from its closing-off position to its injection position, and, on the other hand, to translationally lock the said base with respect to the said sealingly closing-off means in the said injection position.

17. Injection device as claimed in claim 16, wherein the sealingly closing-off means comprise a closing-off stopper made of an impermeable elastic material having the shape of a hollow cylindrical body with a cross-section adapted to be inserted into the receptacle, the said closing-off stopper comprising a back wall for delimiting the chamber of the said receptacle, in which wall a passing-through delivery duct is formed, the said injection device furthermore comprising:
a cover provided with an end wall in which there is an orifice with a diameter adapted to permit the passage of the base, and with a cylindrical sleeve with a diameter adapted to fit together with the closing-off stopper, so that the said sleeve and stopper define a duct for guiding the said base,
means for relative translational locking of the closing-off stopper and the cover,
and means for relative translational locking of the closing-off stopper/cover assembly with respect to the receptacle.

18. Injection device as claimed in claim 17 taken together, wherein the means for translational locking of the base in its injection position comprise at least one radially deformable stop element arranged so as to be kept radially compressed inside the sleeve of the cover, in the closing-off position of the base and during displacement of the latter to its injection position, and to expand into an assigned locking area adapted to accommodate it in the injection position of the base.

19. Injection device as claimed in claim 17, wherein the cap and the cover are produced in one piece and are connected by detachable connection means consisting of a divisible connection extending between the said cap and the end wall of the said cover.

20. Injection device as claimed in claim 17, wherein the cap and the cover consist of two separate parts comprising mating means for assembly by screwing.

21. Injection device as claimed in claim 17, wherein it comprises an annular crown produced in one piece with the cap and connected to the rear end of the latter by a divisible connection, the said annular crown being adapted for accommodation inside the sleeve of the cover, in a translationally fixed position inside the said sleeve.

22. Injection device as claimed in claim 15, wherein the sealingly closing-off means and the base consist of a part, called the closing-off part, in one piece, provided with a section for sealingly closing off the tubular receptacle adapted to delimit, with the said tubular receptacle, a sealed space with a capacity variable as a function of the longitudinal position of the said closing-off part, the supply duct and the delivery duct both being arranged so as to open into the sealed space in the advanced position of the closing-off part, and so that at least one of the said ducts is closed off by the tubular receptacle and isolated from the sealed space, in the retracted position of this closing-off part.

23. Injection device as claimed in claim 14, wherein the tubular receptacle consists of a cylindrical tube.

24. Injection device as claimed in claim 22, wherein the tubular receptacle consists of a cartridge of the type of cartridge for anaesthetising liquid used in dentistry, comprising a neck having an outer enlargement at its end.

25. Injection device as claimed in claim 24 taken together, wherein the closing-off part has a shape adapted to slide and be guided inside the neck of the cartridge.

26. Injection device as claimed in claim 14, wherein it comprises a case for protecting the needle after injection, comprising a tubular sheath adapted to slide along the tubular receptacle, the said sheath and the said tubular receptacle comprising relative-translational-locking means capable of determining an injection position in which the sheath is set back from the injection needle, and a protection position after use in which the sheath accommodates and masks the injection needle.

27. Injection device as claimed in claim 14, wherein the tubular receptacle consists of a cartridge of the type of cartridge for anaesthetising liquid used in dentistry, comprising a neck having an outer enlargement at its end.
